(19) United States
(12) Patent Application Publication
(10) Pub. No.: US 2019/0048321 A1
(43) Pub. Date: Feb. 14, 2019

(54) Macrophage Enrichment of Cancer Stem Cells

(71) Applicant: UNIVERSITY OF SOUTH CAROLINA, COLUMBIA, SC (US)

(72) Inventor: ESMAIEL JABBARI, ROCKVILLE, MD (US)

(73) Assignee: UNIVERSITY OF SOUTH CAROLINA

(21) Appl. No.: 16/057,882

(22) Filed: Aug. 8, 2018

Related U.S. Application Data
(60) Provisional application No. 62/545,196, filed on Aug. 14, 2017.

(51) Int. Cl. C12N 5/095; C12N 5/00

(52) U.S. Cl. CPC C12N 5/0695; C12N 5/0062; C12N 2503/02; C12N 2539/00; C12N 2513/00

(57) ABSTRACT

Disclosed are culture systems and methods that can enrich a cancer cell population in cancer stem cells. The culture system can be utilized for study of cancer stem cells in general as well as for cancer drug screening. The methods and systems include co-culturing of a cancer cell population and programable macrophages, which can lead to proliferation of the cancer stem cells of the population and depletion of differentiated cancer cells of the population. The methods and systems can provide a more reliable and accurate model for preclinical drug toxicity testing.

### CROSS REFERENCE TO RELATED APPLICATION

This application claims filing benefit of U.S. Provisional Patent Application Ser. No. 62/545,196 having a filing date of Aug. 14, 2017, which is incorporated herein by reference for all purposes.

### GOVERNMENT SUPPORT CLAUSE

This invention was made with government support under grant no. 1403545 awarded by the National Science Foundation. The government has certain rights in the invention.

### BACKGROUND

An estimated 12 million new cancer cases are diagnosed worldwide every year with 1.5 million of those new cases in the US alone. Moreover, cancer is estimated to cause 8 million deaths every year worldwide with approximately 500,000 of those occurring in the US. The overall costs of cancer are estimated at $125 billion/year and this could increase to $200 billion by the year 2020. A major contributing factor to mortality in cancer patients as well as treatment costs is recurrence in the form of relapse (after surgery or therapy) and development of resistance to therapy.

Cancer recurrence is related to the existence of cancer stem cells (CSCs) in the tumor tissue. Normal stem cells and CSCs use similar signaling pathways to maintain their ‘sternness.’ However, they are believed to respond to environmental cues differently. Under normal conditions, stem cell proliferation and differentiation is inhibited, but CSCs, due to mutations in the cell, are self-sufficient with respect to proliferation. It has been proposed that in the case of CSCs the stem cell niche supports cell proliferation, as opposed to inhibition of proliferation in normal stem cells. In addition, CSCs exhibit high expression of ATP-binding cassette (ABC) transporter proteins associated with drug resistance and as such show resistance to many therapies that are effective against other cells of the tumor population. Accordingly, though CSCs are present in a very small population in tumor tissue, generally less than 1% of the total tumor cell population, the CSCs that survive after therapy can grow into a heterogeneous population of colonies with a range of invasiveness with high resistance to therapy.

Typically, the bulk of a tumor will shrink to less than 1% of its initial volume after cancer therapy, but the remaining tumor tissue then becomes enriched with CSCs that are highly resistant to future therapies. Consistent with this model, 15% of breast cancer patients are diagnosed with the most aggressive triple negative breast cancer (TNBC) having the highest sub-population of CSCs. These patients show a survival rate of 77% after 5 years, compared to 93% for other breast cancer subtypes.

Newly discovered drugs in the pharmaceutical industry are screened against the entire population of patient's cancer cells or the entire population of cells in a cancer cell line. Since CSCs constitute only a small fraction of the entire cancer cell population, conventional drug screening approaches fail to measure the response of CSCs to the drug. Thus, the drug can pass the toxicity test and move to the next stage of animal and human testing without testing for toxicity against the most invasive sub-population of cells in the tumor tissue. Unfortunately, many drugs that are used in cancer therapy, like Paclitaxel or Doxorubicin, have been shown to be ineffective against the most aggressive CSC sub-population of cancer cells. As a result, tumors relapse after therapy with much higher aggressiveness, often leading to patient death.

What are needed in the art are techniques that can screen drugs against the most invasive sub-population of cancer cells in many different cancer types. In particular, methods and approaches that can enrich the stem cell sub-population of cancer cells are very beneficial to drug screening.

### SUMMARY

According to one embodiment, disclosed is a method for enriching a cancer cell population in cancer stem cells. For example, a method can include encapsulating a cancer cell population in a biocompatible matrix. The cancer cell population can include CSCs, for instance as a component of a cancer cell population obtained from a patient or a cancer cell line. The method can also include encapsulating macrophages in the biocompatible matrix such that the macrophages are in chemical communication with the cancer cell population. For instance, the cancer cell population and the macrophages can be mixed together in a single homogeneous matrix or can be separated in different areas of a heterogeneous matrix, but in either case still in chemical communication with one another. A method also includes culturing the cells for a period of time, during which the cancer cell population becomes enriched in the CSCs.

Also disclosed is a method for testing the efficacy of an agent against CSCs. For instance, following enrichment of a sample in CSCs, an agent of interest, e.g., a known or potential cancer drug, can be combined with the cancer cell population that is enriched in CSCs, and the effect of the agent on the CSCs can be determined.

According to another embodiment, a three-dimensional gel culture system is disclosed that includes a cancer cell population and programmable macrophages encapsulated within a biocompatible matrix and in chemical communication with one another. Optionally, the system can also include one or more biologically active agents as supporting material and/or for examination of the system efficacy, for instance in determination of the effectiveness of a known or potential cancer drug against the CSCs.

### BRIEF DESCRIPTION OF THE FIGURES

A full and enabling disclosure of the present subject matter, including the best mode thereof to one of ordinary skill in the art, is set forth more particularly in the remainder of the specification, including references to the accompanying figures in which:

FIG. 1 is a schematic representation of cancer cells and macrophages encapsulated together in a matrix and treated with a cancer drug.

FIG. 2 presents the cell density for matrices encapsulated with MDA-MB-231 human breast cancer cells alone (MDA), with human spleen-derived macrophages alone (Mac) or with both (MDA+Mac) (a). Also shown are the expression level of cancer stem cell markers CD44 (b), ABCG2 (c), and EGFR (d) for the matrices.

FIG. 3 provides the expression with culture time of pro-inflammatory (M1) markers TNF-α (a), IL-1β (b), and CCR-7 (c) and the expression of anti-inflammatory pro-healing (M2) markers CD206 (d), CCL12 (e), and CCL18 (f) for matrices encapsulated with MDA alone, with Mac alone, or with both MDA+Mac.

FIG. 4 demonstrates the effect of treatment with Paclitaxel (PTX) on cell content (a) and the expression of cancer stem cell markers CD44 (b), ABCG2 (c), and EGFR (d) for matrices encapsulated with MDA alone, with Mac alone, or with both MDA+Mac.

FIG. 5 illustrates a patterned hydrogel encapsulating cancer cells in a first region of the matrix encapsulating tissue associated macrophages in a second region of the matrix.

### DETAILED DESCRIPTION

The following description and other modifications and variations to the presently disclosed subject matter may be practiced by those of ordinary skill in the art, without departing from the spirit and scope of the disclosure. In addition, it should be understood that aspects of the various embodiments may be interchanged in whole or in part. Furthermore, those of ordinary skill in the art will appreciate that the following description is by way of example only, and is not intended to limit the invention.

The present disclosure is generally directed to an inert 3D gel culture system that can enrich a cancer cell population in CSCs, which are generally considered to be the most aggressive sub-population in a population of cancer cells. The gel culture system can be utilized for cancer drug screening against the most invasive cell colony in the population of cancer cells. The methods and systems can provide a more reliable and accurate model for preclinical drug toxicity testing and can significantly decrease preclinical screening expenses while reducing the chance of late-stage failure of the drug candidate in clinical trials.

The CSC enrichment methods include co-culturing of cancer cells and macrophages through co-encapsulation in a three-dimensional matrix. The properties of the matrix and the presence of macrophages can lead to enrichment of the most aggressive colonies of CSCs. Moreover, as the matrix is inert, differentiated cancer cells of a cancer cell population encapsulated in the matrix can be depleted while the CSCs can be enriched. The depletion of differentiated cancer cells and enrichment of stem cell colonies can provide a cell population that is highly enriched in the most aggressive cancer cell population of the initial population. As such, the system can be used in one embodiment to test the efficacy of agents (e.g., prospective treatment agents) against the most aggressive and malignant cells in the population of cancer cells.

Beneficially, the 3D gel culture system can be used with all cancer cells including breast, lung, colon, gastric, liver, thyroid, bladder, oral, ovarian, nasal, just to name a few. The system can be especially beneficial in study of cancers that have a high recurrence rate and/or low survival rate, e.g., triple-negative breast cancer (TNBC), for which many existing treatments have little or no effect on CSCs present in low fractions in the tumor population.

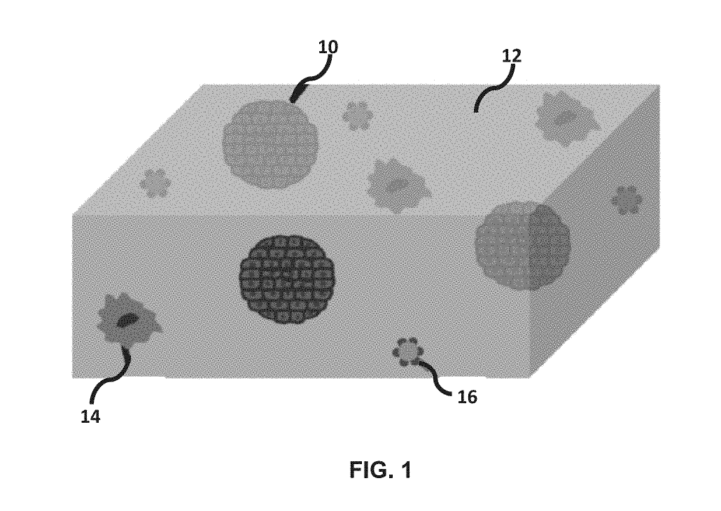

FIG. 1 schematically illustrates a typical system. As shown, macrophages 14 are encapsulated in a 3D matrix 12 so as to stimulate the growth of CSCs, which can be a sub-population of a larger cancer cell population initially encapsulated in the matrix 12, at the expense of differentiated cancer cells of the population. This leads to CSC enrichment and the formation of highly CSC-enriched tumor spheroids 10. Optionally, a cancer drug 16 can be included/added to the matrix 12 for testing against the CSC-enriched sub-population of cancer cells.

A cell population that can be examined can include a single cell type or multiple different cell types combined together, as desired. For instance, in one embodiment CSCs can be encapsulated in a matrix with no other cell types. In general, however, a cell population including differentiated cancer cells (e.g., breast cancer cells, lung cancer cells, etc.) in conjunction with CSCs can be encapsulated in a matrix. For example, ex vivo tumor tissue including differentiated cancer cells and CSCs in a proportion as found in vivo can be supported by the matrix. Of course, non-cancerous cells can also be encapsulated in or supported in a matrix, for instance in conjunction with differentiated cancer cells and CSCs. Other cells can include, without limitation, support cells or tumor stroma that can function as support for the growth of cancer cells. Support cells can include, without limitation, mesenchymal cells, endothelial cells, immune system cells, lymphatic cells, etc.

A system can include programmable macrophages in conjunction with the CSCs. Macrophages include a heterogeneous population of cells found in most tissues of the body. These cells are capable of performing a broad spectrum of functions, one of which is to phagocytose and remove foreign substances and cells expressing receptors that are not normally found on the surface of cells. Programmable macrophages can take on a pro-inflammatory (M1 type) phenotype when substances need to be removed from tissue or an anti-inflammatory (M2 type) or healing phenotype once the substance is removed and the tissue is in the stage of rebuilding. Macrophages present in tumor tissue are referred to as tumor-associated macrophages (TAMs).

Macrophage infiltration has been correlated with severity in many types of cancer. Tumor cells recruit macrophages and educate them to adopt an M2-like phenotype through the secretion of chemokines and growth factors, such as MCP1 and CSF1. Macrophages in turn promote tumor growth through supporting angiogenesis, suppressing anti-tumor immunity, modulating extracellular matrix remodeling, and promoting tumor cell migration. Thus, tumor cells and macrophages interact to create a feedforward loop supporting tumor growth and metastasis.

In addition to these previously known functions of TAMs, there is cross-talk between TAMs and the stem cell sub-population of cancer tissue, which has led to the development of the disclosed methods and systems. Without wishing to be bound to any particular theory, it is believed that the CSCs of the tumor tissue release factors that instruct TAMs to take an anti-inflammatory M2 phenotype and TAMs in turn release factors that stabilize and maintain the stem cell niche. It is this cross-talk that is advantageously utilized in disclosed systems and methods to enrich CSCs in a cancer cell population.

The biocompatible matrix of a system can generally be formed of any synthetic or natural biocompatible polymer (or combination thereof) that does not support adhesion of cancer cells during cultivation and is thus a non-adherent and inert matrix. As utilized herein the term “inert matrix” generally refers to a matrix the presence of which does not interact through specific or non-specific ligand-receptor interactions with the cells encapsulated in the matrix or the presence of which is non-fouling to the proteins secreted by the cells encapsulated in the matrix. For instance, in one embodiment, a biocompatible, inert hydrogel matrix can be based upon poly(ethylene glycol) (PEG). This is not a requirement of the gel systems, however, and other synthetic non-adherent inert and biocompatible polymers can be utilized in conjunction with or alternative to a PEG-based system. For instance, an agarose-based gel can be utilized in one embodiment of a natural polymer-based gel. For instance, a hydroxyl ethyl cellulose can be utilized in another embodiment of a natural polymer-based gel.

The polymer(s) used to form the matrix can have any suitable molecular weight, with the preferred molecular weight depending upon the crosslinking characteristics of the polymer and the targeted elastic modulus of the crosslinked hydrogel matrix. For instance, the polymer can be a low molecular weight polymer having a number average molecular weight of about 1,000 Da or less, a midrange molecular weight polymer having a number average molecular weight of from about 1,000 Da to about 10,000 Da, or a high molecular weight polymer, having a molecular weight of about 10,000 Da or greater. For instance, the polymer can be a di-functional polymer having a molecular weight of about 10,000 Da or less in one embodiment, or from about 1,000 Da to about 5,000 Da in some embodiments.

The particular concentrations of the polymer and/or the crosslinking agent can vary as is known, generally depending upon the gelation characteristics of the polymer. For example, in one embodiment, a precursor solution of a di-functional polymer and crosslinking agent (either prior to or following reaction of the polymer with the crosslinking agent) can have a polymer concentration of about 10% by weight of the solution or less to form a low elastic modulus hydrogel matrix (about 10 kPa or less), a polymer concentration of from about 10% by weight of the solution to about 20% by weight of the solution to form an intermediate elastic modulus hydrogel matrix (about 10 kPa to about 30 kPa), or a polymer concentration of about 20% by weight of the solution or greater to form a high elastic modulus hydrogel matrix (about 30 kPa or greater).

The crosslinking scheme utilized to form the matrix can vary depending upon the particular polymer used in the system. For instance, a crosslinking agent can be reacted with the polymer prior to gel formation or during gel formation, as desired. For example, in one embodiment a polymer (e.g., a PEG polymer) can first be functionalized to form a functional macromer (e.g., a PEG diacrylate) and the functional macromer (e.g., PEGDA) can then be crosslinked to form the gel in the presence of the cell population, for instance by use of an initiator, e.g., a photoinitiator, and subjection to suitable energy (e.g., a UV or visible light cure).

Alternatively, a separate crosslinking agent can be combined with a functionalized polymer at the time of gel formation, and the crosslinking agent can form links between and among the polymers to form the hydrogel network. The crosslinking agent can be a polyfunctional compound that can react with functionality of the polymer to form crosslinks within the hydrogel network. In general, the crosslinking agent can be a biocompatible non-polymeric compound, i.e., a molecular compound that includes two or more reactively functional terminal moieties linked by a bond or a non-polymeric (non-repeating) linking component. By way of example, the crosslinking agent can include but is not limited to diacrylates, di-epoxides, poly-functional epoxides, diisocyanates, polyisocyanates, polyhydric alcohols, water-soluble carbodiim ides, diamines, diaminoalkanes, polyfunctional carboxylic acids, diacid halides, halo acrylate monomers, and so forth. For instance, when considering a PEG-based polymer, a non-polymeric acryloyl halide can be utilized as a crosslinking agent.

In some embodiments, an initiator is utilized to initiate crosslinking of the polymer. Initiators can include photo-initiators, thermal-initiators, or chemical initiators. For example, in one particular embodiment, a UV-initiator can be utilized. Chemical initiators can also be used, such as redox, peroxide, etc. In other embodiments, other radiation initiation processes, such as gamma rays, e-beam, X-ray, etc., can be utilized, which may not require the presence of an initiator.

For example, a non-limiting list of UV-initiators which may be used include IRGACURE® 184 (1-hydroxycyclohexyl phenyl ketone), IRGACURE® 2959 (4-(2-hydroxyethoxy)phenyl-(2-hydroxy-2-methylpropyl)ketone)), and DAROCURE® 1173 (α-hydroxy-α,α-dimethylacetophenone), all commercially available from Ciba Specialty Chemicals (Terrytown, N.Y.).

When present, only one initiator is necessary, however, one or more second initiators may be utilized. The one or more second initiators can be photo or chemical initiators. The amount of initiator can generally be supplied in standard amounts, for instance in the range of about 0.01 to about 5% by weight of the polymer solution

In one embodiment, the matrix can be developed with a predetermined elastic modulus in a well-defined narrow range. This can be very useful as the gel modulus can have a strong effect on tumorsphere formation and the effect can be bimodal. For instance, the elastic modulus of normal human breast tissue is lower than 4 kPa while that of cancerous breast tissue can be up to 40 kPa, and as such the matrix can be formed with an elastic modulus so as to reproduce the microenvironment of a biological system anywhere within this range. For example, the matrix can be designed to have an intermediate elastic modulus of from 10 kilopascals (kPa) to 30 kPa, a low modulus of less than 10 kPa, for instance from 2.5 kPa to 7.5 kPa, or a high modulus of from 10 kPa to 70 kPa. Determination of elastic modulus of a gel can be carried out according to standard practices, for instance by loading a sample of the gel on the Peltier plate of a rheometer and subjecting the gel to uniaxial compressive force. The slope of the linear fit to the stress-strain curve can be taken as the elastic modulus (E) of the gel.

The mesh size of the matrix can generally be from about 25 nanometers (nm) to about 95 nm, for instance from about 35 nm to about 70 nm. The mesh size can be calculated from the modulus and equilibrium swelling ratio of the gels using the Peppas and Barr-Howell equation, as is known in the art. For instance, as the macromer concentration of a precursor solution increases from 7.5% to 10%, to 15%, to 20%, and to 25%, the mesh size can decrease from 93±4 nm to 67±3, 53±3, 34±2, and 25±2 nm, respectively.

Cells can generally be combined with a matrix precursor solution prior to crosslinking of the matrix. For instance, a cancer cell population obtained from a patient or a cancer cell line can be added to the hydrogel precursor solution under sterile condition and mixed to form a uniform suspension. Cancer cell loading can generally be in the range of about 10,000 cells/mL to about 10,000,000 cells/m L.

Any cancer cell type or any cancer cell line may be used including but not limited to MDA-MB-231 human breast cancer cells, MCF7 human breast cancer cells, 4T1 mouse breast cancer cells, HCT116 human colon cancer cells, AGS human gastric cancer cells, and U2OS human osteosarcoma cells, to name a few. Cancer cells from different species including, without limitation, human, mouse, and rat may be used. Tumor cells harvested from cancer patients may be used, generally following digestion of the tissue and isolation of the tumor cells.

Macrophages can be combined with the cancer cells in the matrix or encapsulated in a second region of a heterogeneous matrix. In either case, however, the macrophages can be in chemical communication with the cancer cells. For example, in one embodiment, macrophages can be added to a hydrogel precursor solution under sterile conditions with the cancer cells and the combination can be mixed to form a homogenous suspension including both cancer cells and macrophages in the precursor solution.

In another embodiment, cancer cells and macrophages can be separately encapsulated in matrices that can then be held in chemical communication with one another. For example, cancer cells can be suspended in a first hydrogel precursor solution and macrophages can be suspended in a second hydrogel precursor solution. The two precursor solutions can be the same or different from one another, e.g., can include the same polymers and crosslinking functionalities in the same concentrations, the same support materials, etc. or alternatively, one or more aspects of the precursor solutions and/or the crosslinked matrices can differ. For instance, the first hydrogel precursor solution that includes a suspension of cancer cells can be crosslinked to form a matrix with a relatively high elastic modulus, similar to that of cancerous tissue, while the second hydrogel precursor solution that includes a suspension of macrophages can be crosslinked to form a matrix with a lower elastic modulus, similar to that of healthy, non-cancerous tissue.

To hold the cancer cells and macrophages in chemical communication with one another, the matrices can be crosslinked adjacent to one another. For instance, a photomask defining a pattern (e.g., one or more circles, squares, etc.) can be placed on top of a layer of the precursor solution and then the solution can be crosslinked in the areas defined by the mask to provide a patterned gel encapsulating the cancer cells.

The size and separations between individual pattern components can allow for chemical communication between the cancer cells held in a first component and macrophages held in a second component. For instance, an individual segment of pattern can range in cross-sectional dimension from about 50 μm to about 500 μm and the separation distance between individual patterned components can generally range from about 100 μm to about 500 μm.

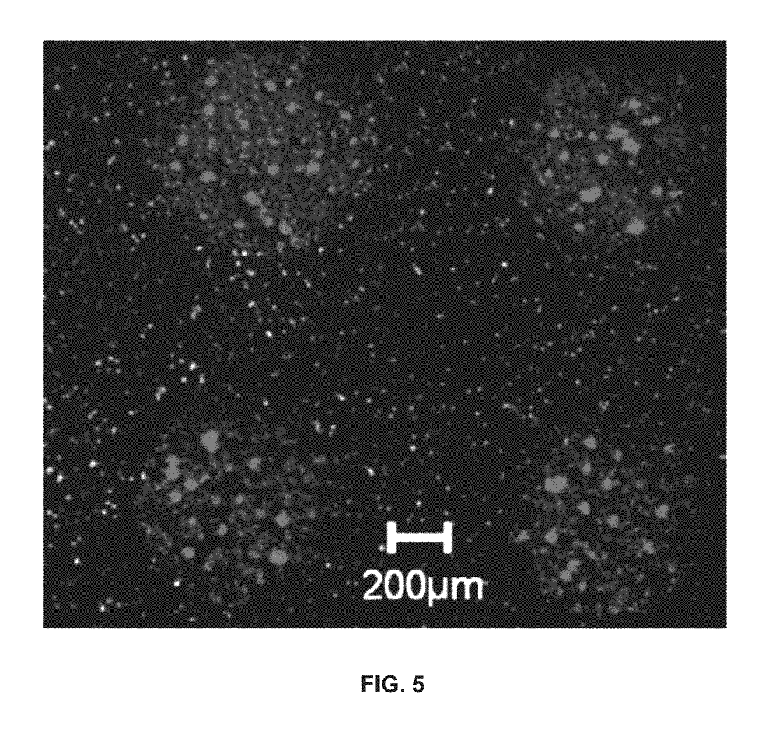

Following crosslinking of the first matrix (optionally forming a plurality of pattern elements), any remaining solution that has not been crosslinked can be washed away. Following, the second hydrogel precursor solution within which the macrophages are suspended can be loaded adjacent to the cancer cell-containing patterned elements and this solution can then be crosslinked to form the matrix elements encapsulating the macrophages. The formed matrix can then be heterogeneous with some areas encapsulating the cancer cells and other adjacent areas encapsulating the macrophages, but with the two cell types held in chemical communication with one another. One example of such a heterogeneous matrix is illustrated in FIG. 5 and the formation of this matrix is described in the Examples section.

In yet another embodiment, a heterogeneous matrix can include one or more areas of cancer cells mixed with macrophages as well as one or more areas of cancer cells alone and/or one or more areas of macrophages alone.

The macrophage loading (whether mixed with the cancer cells or in an adjacent area) can generally be in the range of about 10,000 cells/mL to about 10,000,000 cells/m L. Any programmable macrophage that can undergo phenotypic change from pro-inflammatory M1 to anti-inflammatory M2 may be used including but not limited to Human CRL-9850 macrophages harvested from spleen (ATCC, Manassas, Va.), Abelson mouse RAW 264.7 macrophages (ATCC TIB-71), mouse J774A.1 (ATCC TIB-67), and rat alveolar NR8381 macrophages (ATCC CRL-2192) to name a few. Macrophages from different species including but not limited to human, mouse, and rat may be used. Macrophages harvested from healthy or cancer patients may be used. For instance, in one embodiment, both the cancer cell population and the macrophages can be obtained from a single subject.

A matrix can include additional materials that can improve culturing and/or observation of the system. For instance, a cell-adhesive agent like an RGD peptide may be added to a precursor solution to improve cell adhesion in a matrix, in particular the adhesion of macrophages to the matrix, which can use an adherent gel for growth. In those embodiments in which a heterogeneous matrix is formed, an adherent such as RGD would generally not be used in the portion of the gel that encapsulates the cancer cells, and the cancer cells can be encapsulated in the non-adherent and inert gel.

Following formation, the cancer cells and the macrophages can be cultured in suitable cell culture medium for a time of about 3 days or more, for instance from about 3 to about 14 days. During the culture period, differentiated cancer cells of the initial cell population can undergo apoptosis or remain in GO phase in the matrix whereas the CSCs can proliferate and develop and form CSC spheres, leading to the enrichment of the CSC sub-population of cancer cells. It is believed that the macrophages in the matrix enhance the maintenance of stem cells and increase the probability of CSC self-renewal, thus leading to their further enrichment. Conversely, CSCs, through cell-cell contact and/or growth factor release, can alter the phenotype of macrophages from pro-inflammatory to anti-inflammatory which can lead to further stabilization and enrichment of CSCs over differentiated cancer cells.

Disclosed systems and methods can be utilized to screen a biologically or chemotherapeutically active agent for effectiveness against CSCs. In one particular embodiment, a system can be utilized in screening a cancer drug for efficacy. Any cancer drug and any type of cell population are encompassed herein. As utilized herein, the term “cancer drug” generally refers to any agent useful to combat cancer. A non-limiting list of cancer drugs that can be investigated by use of the disclosed matrices can be found in, for example, U.S. Pat. No. 5,037,883, which is incorporated herein by reference. U.S. Pat. Nos. 6,348,209, 6,346,349, and 6,342,221 also describe agents related to cancer drugs, all of which are incorporated herein by reference.

Classes of cancer drugs encompassed herein include, but are not limited to, chemotherapeutic agents, cytotoxins, antimetabolites, alkylating agents, protein kinase inhibitors, anthracyclines, antibiotics, antimitotic agents (e.g. anti-tubulin agents), corticosteroids, radiopharmaceuticals, antibodies, and proteins (e.g. cytokines, enzymes, or interferons). Cancer drugs can include, for example, small molecule organic compounds, macromolecules, metal containing compounds, and compounds or chelates that include radionuclides. In example embodiments, the cancer drug can be a small molecule organic compound. Specific examples include, but are not limited to docetaxel, gemcitabine, imatinib (Gleevec®), 5-fluorouracil, 9-aminocamptothecin, amine-modified geldanamycin, doxorubicin, paclitaxel (Taxon, cisplatin, procarbazine, hydroxyurea, meso e-chlorin, Gd(+3) compounds, asparaginase, and radionuclides (e.g., I-131, Y-90, In-111, and Tc-99m). There are many cancer drugs known in the art and many continue to be developed. In some embodiments, two or more cancer drugs can be examined simultaneously.

Disclosed methods and systems are not limited to the screening of cancer drugs, however, and can be utilized in any suitable application. For instance, the disclosed matrices can be utilized to selectively enrich or deplete a cell population in CSCs so as to better understand the development of tumors in the cell population, to examine the effects of potential treatment protocols on the cell population, and particularly on CSCs of a cell population, and so forth.

The presently disclosed subject matter may be better understood with reference to the Examples set forth below.

### EXAMPLE 1

Polyethylene glycol was functionalized with acrylate end-groups by reaction with acryloyl chloride using well-established procedures to generate a photopolymerizable polyethylene glycol diacrylate (PEGDA). The PEGDA and a photoinitiator (4-(2-hydroxyethoxy)phenyl-(2-hydroxy-2-propyl) ketone (commercial name Irgacure-2959)) were dissolved in aqueous solution to generate a hydrogel precursor solution. Prior to cure, the hydrogel precursor solution was combined with MDA-MB-231 human breast cancer cells alone (MDA), with human spleen-derived macrophages alone (Mac) or with both (MDA+Mac).

To form the matrices, the cancer cells and/or macrophages were suspended in the PEGDA hydrogel precursor solution, the suspension was pipetted in a tissue culture plate as a thin film, and the film was photopolymerized with UV radiation to encapsulate the cells in the hydrogel matrix. The matrices had a stiffness of 5 kPa matrix.

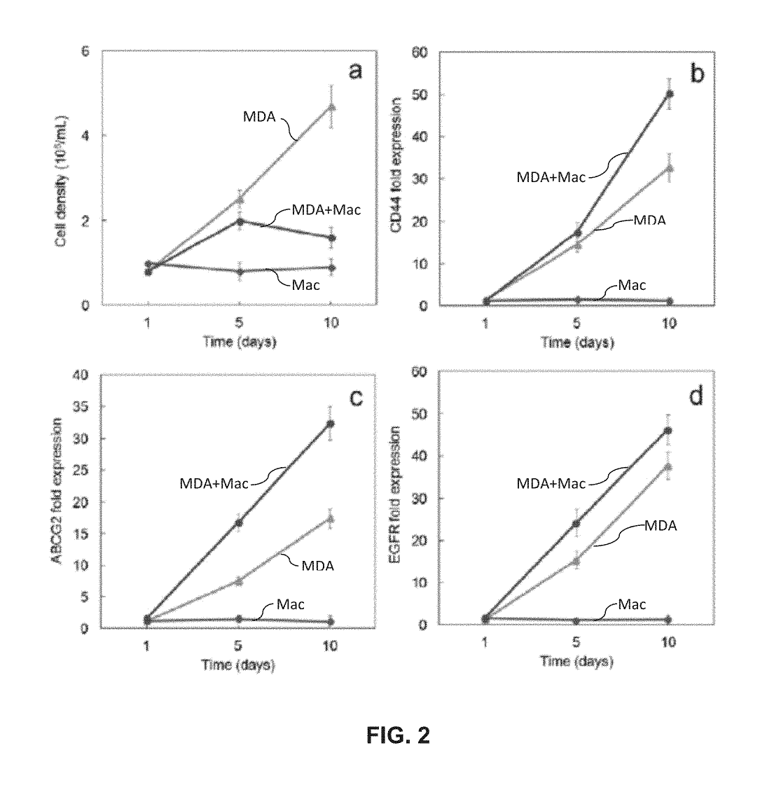

FIG. 2 presents the cell density with time for matrices encapsulated with either MDA-MB-231 human breast cancer cells alone (MDA), with human spleen-derived macrophages alone (Mac) or with both (MDA+Mac).

Also shown in FIG. 2 is the expression level of cancer stem cell markers CD44 (b), ABCG2 (c), and EGFR (d). As can be seen, when cultured alone in a matrix macrophages (Mac) did not grow and there was no expression of CSC markers as macrophages do not express markers of CSCs. MDA cancer cells cultured alone (MDA) grew in the matrix by forming spheres with mixed population of stem and differentiated cells and as shown the growing cells express CSC markers. For MDA cancer cells co-cultured with macrophages (MDA+Mac), the cell number was lower than MDA alone with incubation time but the expression of CSC markers was higher. As can be seen for the MDA+Mac co-culture, the cell number initially increased followed by a decline. However, there was a dramatic increase in the expression of CSC markers following the decline in cell number for the co-culture, leading to further enrichment of CSC albeit lower cell content.

FIG. 2 indicates that the tumor spheres formed in the presence of macrophages had a higher fraction of stem cells as compared to differentiated cells, leading to the conclusion that the macrophages shifted the symmetry of cell division toward stem cells and self-renewal. Thus, macrophages had a stabilizing effect on CSCs and maintained the CSC niche.

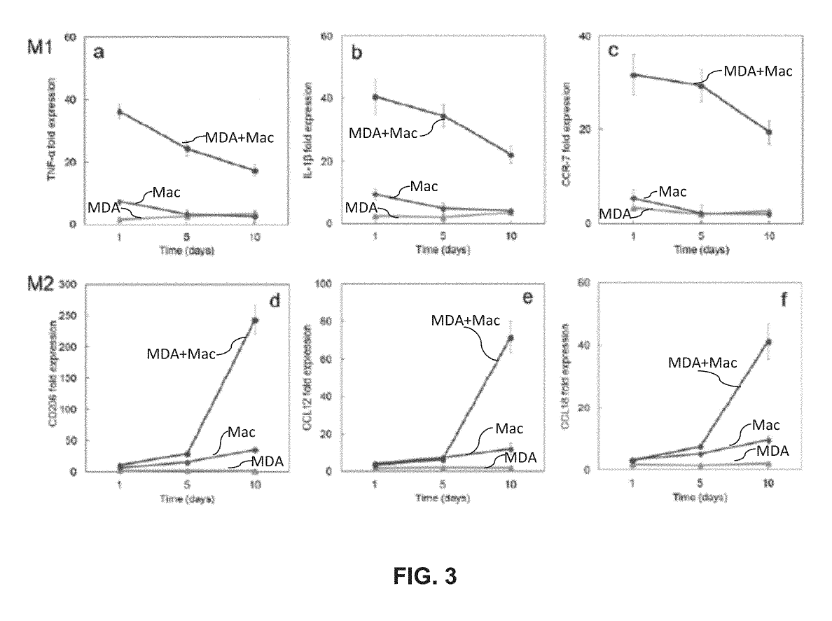

FIG. 3 illustrates the expression with time of pro-inflammatory (M1) markers TNF-α (a), IL-1β (b), and CCR-7 (c) and the expression of anti-inflammatory pro-healing (M2) markers CD206 (d), CCL12 (e), and CCL18 (f) for the different matrices. As shown, when MDA cancer cells were cultured alone in the matrix, there was no expression of M1 or M2 markers with incubation time. Macrophages alone had a low expression of M1 markers initially which decreased to no expression with incubation time. Further, macrophages alone had no expression of M2 markers initially which increased slightly with incubation time. Macrophages co-cultured with MDA cancer cells initially had very high expression of M1 markers and low expression of M2 markers, indicating that the presence of whole population of cancer cells induced expression of pro-inflammatory markers of macrophages; however, as CSCs grew and formed tumor spheres and CSC fraction in the matrix increased, the expression of M1 markers decreased with incubation time (yet it remained high) and the expression of anti-inflammatory M2 markers sharply increased. These results indicate that CSCs played a role in shifting the phenotype of macrophages from pro-inflammatory to anti-inflammatory in the tumor microenvironment.

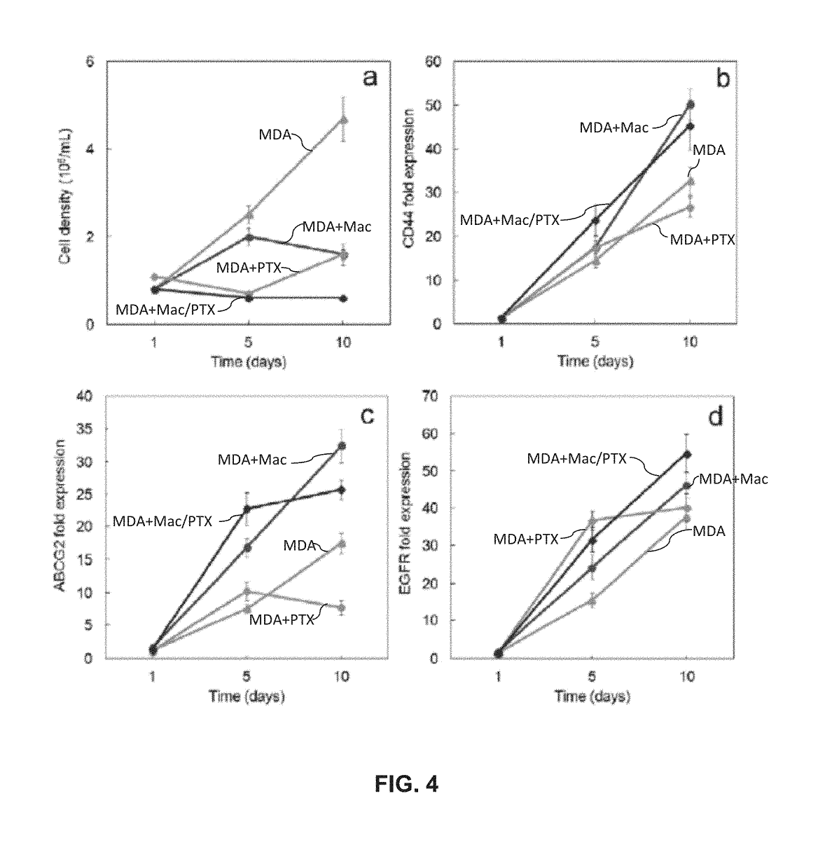

This co-culture system was also used to screen Paclitaxel (PTX) toxicity against CSCs. Paclitaxel has been shown to be effective against MDA-MB-231 cells in previous studies in 2D tissue culture plates. FIG. 4 demonstrates the effect of treatment of the different matrices with Paclitaxel (PTX) on cell content (a) and the expression of cancer stem cell markers CD44 (b), ABCG2 (c), and EGFR (d). MDA cancer cells cultured alone with no PTX (MDA) grew by forming tumor spheres with mixed population of stem and differentiated cells and as shown the growing cells expressed CSC markers. For MDA cancer cells co-cultured with macrophages and no PTX (MDA+Mac) and for MDA cancer cells treated with PTX (MDA+PTX), the cell number increased somewhat and the expression of CSC markers increased significantly so it is unclear if PTX was effective against CSCs. For MDA cancer cells with macrophages treated with PTX (MDA+Mac/PTX), the expression of CSC markers (CD44, ABCG2, EGFR) sharply increased with incubation as the drug halted the growth of differentiated cancer cells. Although PTX was effective in halting cell growth and the cell content decreased, there was a cell sub-population with very high expression of CSC markers that remained, thus PTX was shown to be ineffective against CSCs in MDA-MB-231 breast tumor cells.

### EXAMPLE 2

A patterned hydrogel was formed encapsulating cancer cells in a first region of the matrix and encapsulating tissue associated macrophages in a second region of the matrix. To form the patterned matrix, MDA-MB-231 cancer cells were suspended in a first PEGDA hydrogel precursor solution and macrophages were suspended in a second PEGDA hydrogel precursor solution. The solutions were identical save for the cell type suspended therein. Next, a layer of the first hydrogel precursor solution including the MDA cells was pipetted on a glass plate. Then, a photomask with circular patterns was placed on top of this hydrogel layer and crosslinked to form a patterned gel encapsulating the cancer cells. The plate was then washed with PBS to remove the uncrosslinked solution.

Following, the second hydrogel precursor solution that included the macrophages was added to the plate to fill the empty volumes between the patterns and this second precursor solution was then crosslinked to form a matrix encapsulating macrophages. This approach resulted in patterns of cancer cells in a matrix of macrophages.

FIG. 5 illustrates the resulting patterned matrix. The lighter circular sections were stained to show the MDA cancer cells and the surrounding darker sections were stained to show the macrophage-containing sections.

It will be appreciated that the foregoing examples, given for purposes of illustration, are not to be construed as limiting the scope of this disclosure. Although only a few exemplary embodiments have been described in detail above, those skilled in the art will readily appreciate that many modifications are possible in the exemplary embodiments without materially departing from the novel teachings and advantages of this disclosure. Accordingly, all such modifications are intended to be included within the scope of this disclosure which is defined in the following claims and all equivalents thereto. Further, it is identified that many embodiments may be conceived that do not achieve all of the advantages of some embodiments, yet the absence of a particular advantage shall not be construed to necessarily mean that such an embodiment is outside the scope of the present invention.

1. A method for culturing a population of cancer cells, the method comprising:
encapsulating a cancer cell population in a biocompatible matrix, the cancer cell population including cancer stem cells, wherein at least a portion of the biocompatible matrix is non-adherent to the cancer stem cells and the differentiated cancer cells, and wherein the biocompatible matrix is inert to the cancer stem cells and to the differentiated cancer cells;
encapsulating macrophages in the biocompatible matrix, the cancer cell population and the macrophages being in chemical communication with one another; and
culturing the cancer cell population and the macrophages while held in chemical communication with one another in the biocompatible matrix, wherein over the course of the culturing, the cancer cell population is enriched in the cancer stem cells and depleted in the differentiated cancer cells.

2. The method of claim 1, the differentiated cancer cells comprising breast, lung, colon, gastric, liver, thyroid, bladder, oral, ovarian, or nasal cancer cells.

3. The method of claim 1, further comprising deriving the cancer cell population from a subject.

4. The method of claim 1, further comprising obtaining the cancer cell population from a cancer cell line.

5. The method of claim 1, the method comprising culturing the cancer cell population and the macrophages while held in chemical communication with one another in the biocompatible matrix for a period of time of about 3 days or more.

6. The method of claim 5, the method further comprising following the period of time, combining a biologically active agent with the cancer cell population.

7. The method of claim 6, wherein the biologically active agent is a cancer drug or a potential cancer drug.

8. The method of claim 1, the method further comprising encapsulating the cancer cell population in a first area of the biocompatible matrix and encapsulating the macrophages in a second area of the matrix, the first and second areas being adjacent to one another, the first area of the biocompatible matrix being non-adherent to the cancer stem cells and the differentiated cancer cells.

9. The method of claim 8, the second area of the matrix comprising a cell-adhesive agent.

10. The method of claim 1, wherein at encapsulation, the cancer cell population has a total cancer cell concentration of from about 10,000 cells/mL to about 10,000,000 cells per milliliter.

11. The method of claim 1, wherein at encapsulation the macrophages are encapsulated at a concentration of from about 10,000 cells/mL to about 10,000,000 cells per milliliter.

12. A cancer cell culture system comprising:
a three-dimensional biocompatible matrix, wherein at least a portion of the biocompatible matrix is inert and non-adherent to cancer cells;
a cancer cell population encapsulated in the matrix, the cancer cell population comprising cancer stem cells and differentiated cancer cells;
macrophages encapsulated in the matrix, wherein the cancer stem cells are in chemical communication with the macrophages.

13. The cancer cell culture system of claim 13, the differentiated cancer cells comprising breast, lung, colon, gastric, liver, thyroid, bladder, oral, ovarian, or nasal cancer cells.

14. The cancer cell culture system of claim 12, the cancer cell population and/or the macrophages comprising ex vivo cells.

15. The cancer cell culture system of claim 12, the system further comprising non-cancerous cells encapsulated in the biocompatible matrix.

16. The cancer cell culture system of claim 12, the biocompatible matrix comprising a crosslinked poly(ethylene glycol).

17. The cancer cell culture system of claim 12, the biocompatible matrix having an elastic modulus of from about 1.0 kilopascals to about 70 kilopascals.

18. The cancer cell culture system of claim 12, wherein the cancer cell population is encapsulated in a first area of the biocompatible matrix and the macrophages are encapsulated in a second area of the matrix, the first and second areas being adjacent to one another, the first area of the biocompatible matrix being non-adherent to the differentiated cancer cells and to the cancer stem cells.

19. The cancer cell culture system of claim 17, wherein a characteristic of the biocompatible matrix in the first area differs from the characteristic of the biocompatible matrix in the second area.

20. The cancer cell culture system of claim 18, wherein the characteristic comprises elastic modulus of the biocompatible matrix.

21. The cancer cell culture system of claim 18, wherein the characteristic comprises the presence of a cell-adhesive agent.
